(19) United States
(12) Patent Application Publication
(10) Pub. No.: US 2013/0079808 A1
(43) Pub. Date: Mar. 28, 2013

(54) ARTERIAL TAMPONADE DEVICE

(71) Applicants: Patrick J. Fitzgerald, Poway, CA (US); R. Todd McKinney, Rancho Santa Fe, CA (US); John A. Simpson, Carlsbad, CA (US)

(72) Inventors: Patrick J. Fitzgerald, Poway, CA (US); R. Todd McKinney, Rancho Santa Fe, CA (US); John A. Simpson, Carlsbad, CA (US)

(73) Assignee: SINOCCLUSIVE LLC, Rancho Santa Fe, CA (US)

(21) Appl. No.: 13/607,482

(22) Filed: Sep. 7, 2012

Related U.S. Application Data
(60) Provisional application No. 61/539,053, filed on Sep. 26, 2011.

(51) Int. Cl. A61M 29/00

(52) U.S. Cl. 606/198

(57) ABSTRACT

An arterial tamponade device has an elongate connecting member of resiliently deformable material with pressure pads at each end of the member, and is expandable between a generally U-shaped or V-shaped collapsed condition and a fully expanded position. The device is inserted into a body cavity in its collapsed state and is released at a predetermined location so that opposite ends of the device are biased away from one another and the pressure pads engage and apply pressure to opposite wall areas of the body cavity before the device is fully expanded. One pressure pad has a bulbous end portion angled away from the connecting member and designed to engage and apply pressure to a predetermined tissue area which includes one or more blood vessels so as to occlude or partially occlude the vessel or vessels and reduce or cut off blood flow to the body cavity.

### RELATED APPLICATION

The present application claims the benefit of co-pending U.S. provisional patent application No. 61/539,053 filed on Sep. 26, 2011, the contents of which are incorporated herein by reference in their entirety.

### BACKGROUND

1. Field of the Invention

The present invention relates generally to tamponade devices used to block or restrict blood flow, and is particularly concerned with an intranasal tamponade device to reduce nasal bleeding by applying force to one or more blood vessels to partially or completely occlude blood flow.

2. Related Art

Bleeding during a surgical procedure is a problem for the surgeon because it limits visibility, complicates the surgery and can add to overall procedural time. This is particularly true in endonasal surgeries, both due to the fact that the nasal cavity is already confined, and the fact that the nasal cavity is highly vascular and small amounts of bleeding obscure the surgical field. Current standards to reduce bleeding during surgery include: a) topical and local administrated medications, b) controlled hypotension, c) use of pre-operative medications, and d) atraumatic surgical techniques. Post-operative bleeding is also often controlled by nasal packing with absorbable and non-absorbable materials. Despite these measures, intra-operative hemostasis remains a significant challenge.

The sphenopalatine artery, an indirect branch of the external carotid artery, is responsible for the majority of blood flow to the nasal cavity and sinus tissues.

There is therefore a need for a mechanism to reduce or eliminate intra-operative bleeding through the sphenopalatine artery, particularly during endonasal surgery.

### SUMMARY

Embodiments described herein provide for a tamponade device to reduce bleeding during and after surgery or to reduce bleeding as a result of other causes.

According to one embodiment, a tamponade device is provided for engagement in a human nasal cavity and applying pressure against a predetermined area of a lateral nasal wall including the sphenopalatine artery (SPA). The device includes an elongate member, an arterial compressor, and a support section. The elongate member includes a central portion, an arterial end portion and a support end portion. The arterial end portion extends from one end of the central portion. The arterial end portion includes a first surface. The support end portion extends from an opposite end of the central portion. The arterial compressor is located at the arterial end portion. The arterial compressor includes an engagement end extending from the first surface. The engagement end is configured to seek and engage a depression in the SPA foramen which houses the SPA. The support section is located at the support end portion. The support section includes a relatively flat tissue engaging surface configured to engage an opposing portion of the septum.

In one embodiment the arterial end portion extends at an angle between zero and forty-five degrees relative to the central portion and the support end portion extends at an angle between zero and forty-five degrees relative to the central portion. In another embodiment the member includes a pair of first guides located on an edge of the central portion. One of the pair of first guides is located adjacent the arterial end portion and the other of the pair of first guides is located adjacent the support end portion. The member also includes a pair of second guides. One of the pair of second guides is located on an edge of the arterial end portion adjacent the central edge portion and the other of the pair of second guides is located on an edge of the support end portion adjacent the central edge portion.

In another embodiment, the enlarged foot or pressure pad configured to apply pressure to one or more arteries may have tissue engaging, bulbous portion or rounded outer end feature extending outwardly at an angle to the connecting member in the compressed configuration. This embodiment is particularly useful as an intranasal arterial tamponade device with the bulbous, outwardly angled portion of the pad positioned to reach into a depression of the SPA (sphenopalatine artery) foramen which houses the SPA and apply pressure on the SPA and tissue overlying the SPA.

The dimensions and material of the elongate connecting member or support frame are such that the feet or pads can be deformed inwardly towards one another with the elongate member compressed into a U or V-like shape for insertion into a body cavity, for example using a medical grasping tool, a tubular sheath, an endoscope or a customized delivery system. When in the proper position, the tool or sheath can be withdrawn or the device can be pushed out of the sheath so that the elongate member deploys, forcing the feet apart into engagement with opposing regions of body tissue in the body cavity. The device is positioned so that the enlarged arterial compressor at the one end of the device engages a key point or area of the body that includes one or more arteries or other blood vessels, applying pressure to occlude the blood vessel or vessels and reduce or eliminate blood supply to the body region involved. The opposite end or support section engages an opposite area of the tissue so as to hold the device in place.

In one embodiment, the elongate connecting member or frame may be made from a shape memory metal, other metal or plastic material suitable for use in the body, such as nitinol (nickel and titanium alloy) or the like. The feet or pressure pads may be of a suitable biocompatible material such as plastic, fabric or other material. The arterial pressure pad may have an end portion injection molded around the end of the bent wire, ribbon or other material.

The connecting member in one embodiment has a central portion and angled end portions to which the pressure pads are attached. This allows for better visualization during insertion of the compressed device. In one embodiment, intranasal tamponade devices with pressure pads oriented in opposite directions are provided for insertion in the right and left nostrils.

The device can help to reduce bleeding during or after surgery, bleeding as a result of injury, or as result of certain medical conditions, by occluding the SPA which provides blood supply to the area involved. In one embodiment the tamponade device is designed as an intranasal arterial tamponade device with the pressure pad on one leg positioned to occlude the SPA at the emergence of the artery from the lateral wall of each side of the nasal cavity. The SPA passes through the sphenopalatine foramen into the cavity of the nose, near the posterior, middle, or superior meatus. The device can be inserted into the nasal cavity, positioned over the sphenopalatine foramen and released such that the device braces against the nasal septum on one end, and engages and places pressure upon the SPA as it emerges from the sphenopalatine foramen on the other end. It can be positioned low in the nose out of the typical endonasal surgical field, using a nasal endoscope device. The feet or pressure pads may be designed with a non-slip outer surface texture, for example with a roughened surface or a surface with plural dimples or bumps, to assist in holding the device in place, in addition to the spring pressure applied by the legs. Once placed in position, the non-slip texture of the feet, combined with the spring force applied by the flexible legs of the device, supports the feet in position while occluding or at least partially occluding the underlying artery. In certain anatomies, the SPA may exit the foramen after having divided into multiple branches.

In one embodiment, the feet can be drug-eluting so as to slowly release a drug over time, for example a drug which further reduces bleeding or which combats inflammation and infection. For example, the feet in one embodiment have a coating of material which holds and elutes a drug into the tissue which they engage, similar to known drug-eluting stents.

The arterial tamponade device described above is particularly useful as an intraoperative device to reduce bleeding in the operative field so as to provide improved visualization of the surgical site, and also to reduce procedural time and associated expense, particularly for endoscopic endonasal procedures which generally produce significant bleeding. The device may also be used to reduce bleeding as a result of trauma or medical conditions such as nose bleed. One known treatment for nose bleeds, as an alternative to lengthy periods of nasal packing, is permanent vascular ligation (via electro-cautery or surgical clips) or invasive arterial embolization which permanently blocks blood flow. Instead of permanent closing of an artery, the tamponade device could be installed to block blood flow temporarily, and then removed once bleeding is under control.

Another application and advantage of the arterial tamponade device is to temporarily anesthetize the posterior nasal cavity through pressure upon the primary nerve that runs in parallel to the SPA in the sphenopalatine foramen. Pressure exerted upon this nerve by the device will likely cause the nerve to temporarily stop functioning, providing a beneficial, transient anesthetic effect during endonasal procedures.

Other features and advantages of the present invention will become more readily apparent to those of ordinary skill in the art after reviewing the following detailed description and accompanying drawings.

### BRIEF DESCRIPTION OF THE DRAWINGS

The details of the present invention, both as to its structure and operation, may be gleaned in part by study of the accompanying drawings, in which like reference numerals refer to like parts, and in which:

FIG. 1A is a bottom plan view of one embodiment of an intranasal arterial tamponade device for insertion in a left nasal cavity, with the device shown in a relaxed, expanded condition;

FIG. 1B is a bottom plan view similar to FIG. 1A, but illustrating a modified device configured for insertion in a right nasal cavity;

FIG. 2 is a longitudinal cross sectional view on the lines 2-2 of FIG. 1A;

FIG. 3 is a front elevation view of the device of FIG. 1 in a compressed condition ready for insertion into a body cavity such as a nasal cavity;

FIG. 4 is a perspective view of the elongate connecting member or frame of the devices of FIGS. 1A to 3 prior to attachment of the arterial compressor and the support section;

FIG. 5 is a side elevation view of the arterial compressor of FIGS. 1A and 1B separate from the connecting member of FIG. 4;

FIG. 6 is a bottom plan view of the arterial compressor of FIG. 5;

FIG. 7 is a coronal cross-sectional view through a nasal cavity illustrating placement of the tamponade device of FIGS. 1A to 6 to occlude the sphenopalatine artery; and

FIGS. 8A to 13 illustrate some alternative embodiments of intranasal arterial tamponade devices.

### DETAILED DESCRIPTION

Certain embodiments as disclosed herein provide for a tamponade device configured for placement between opposing areas of a body cavity so as to apply pressure against a predetermined area of tissue, such as a wall of a cavity in the body including one or more blood vessels, so as to temporarily occlude the blood vessel and stop or reduce blood flow through the vessel or vessels. In one embodiment, the device is designed for placement in a human nasal cavity to occlude at least the sphenopalatine artery (SPA) supplying blood to the nose.

After reading this description it will become apparent to one skilled in the art how to implement the invention in various alternative embodiments and alternative applications. However, although various embodiments of the present invention will be described herein, it is understood that these embodiments are presented by way of example only, and not limitation.

Although the following description and the accompanying drawings are directed to an intranasal arterial tamponade device which is configured for placement in a left or right nostril for exerting pressure on the SPA which supplies blood to the nose, it will be understood that the device may be of appropriate shape and dimensions for placement in other parts of the body or body cavities to apply pressure against blood vessels in tissue, such as cavity walls, so as to temporarily occlude the vessel or vessels or at least reduce blood flow through the vessel and reduce bleeding as a result of surgery, trauma or the like, as discussed in more detail below.

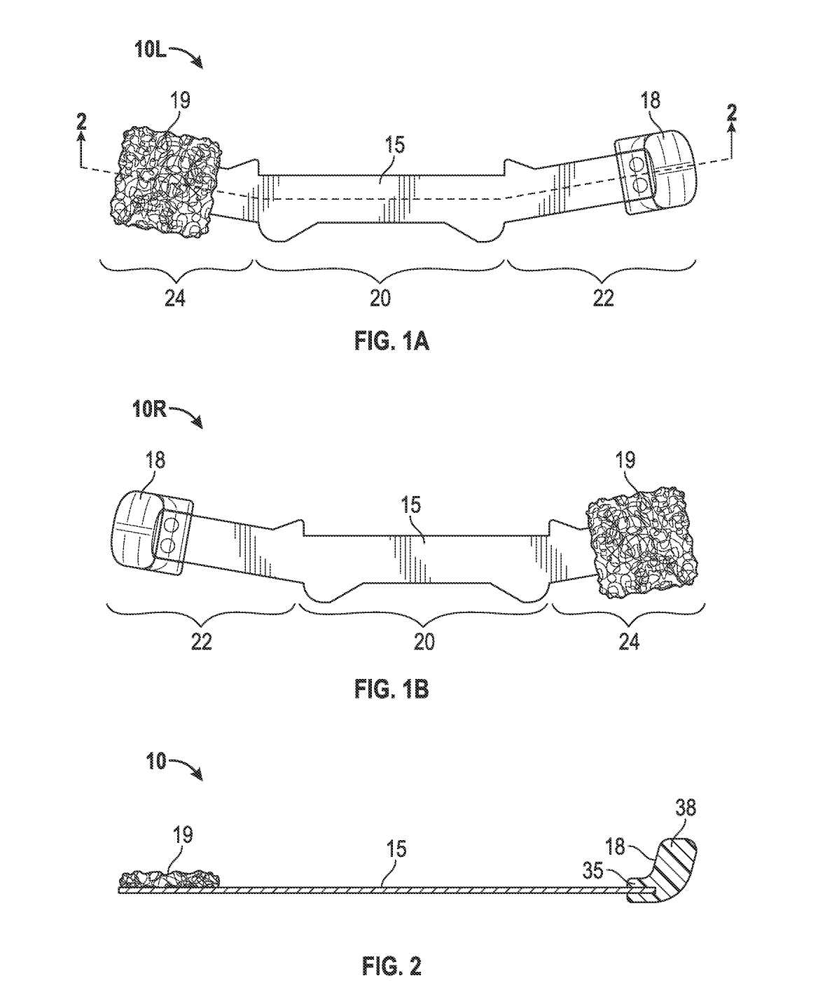

FIGS. 1A to 3 illustrate embodiments of an intranasal arterial tamponade device 10 which is designed to temporarily occlude one or more blood vessels, particularly the SPA in the left or right nasal cavity. FIGS. 1A, 2 and 3 illustrate an embodiment of a nasal cavity device 10L configured for placement in a left nasal cavity, while FIG. 1B illustrates an embodiment of a nasal cavity device 10R configured for placement in a right nasal cavity. The components of the two embodiments are identical, and like reference numbers are used for like components and component parts as appropriate, with the only difference between devices 10L and 10R being the orientation of the arterial compressor 18 and the support section 19 on the connecting member 10, as discussed in more detail below.

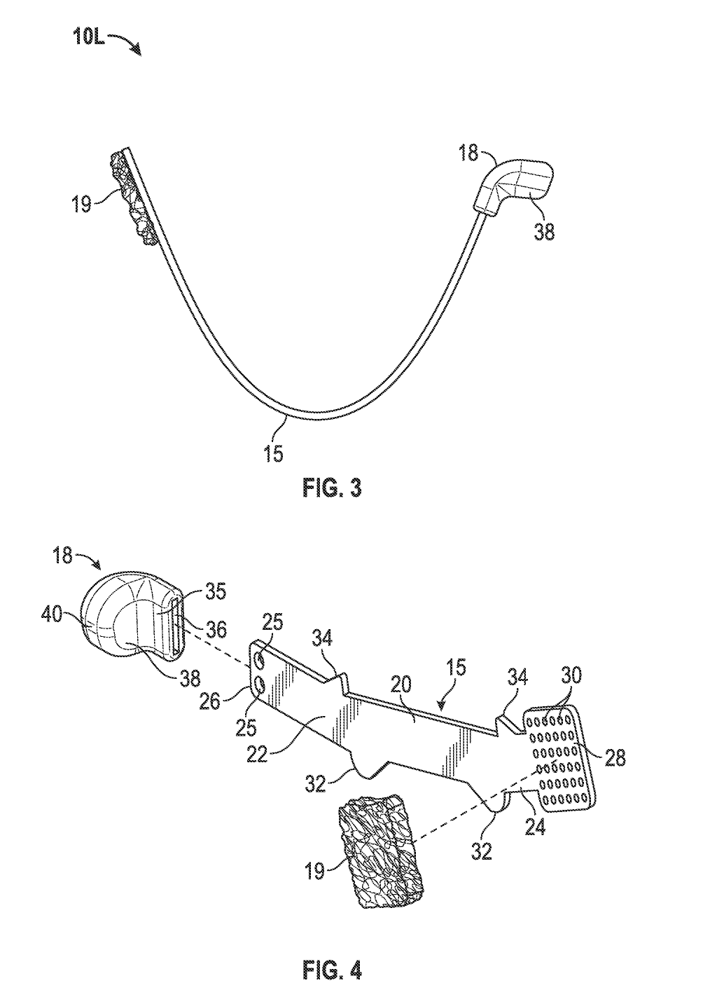

FIGS. 1A and 1B illustrate the left and right intranasal tamponade devices 10L and 10R in a relaxed, fully expanded condition, while FIG. 2 is a longitudinal cross sectional view of the device and FIG. 3 illustrates the left intranasal tamponade device 10L in a partially folded condition ready for insertion into a body cavity such as nasal cavity 14 (as illustrated in FIG. 7). Device 10L includes member 15, arterial compressor 18, and support section 19.

Member 15 may be a relatively thin elongate member or flat strip of resilient or shape-memory material. Member 15 may be formed from a length of a suitable springy or resilient metal or plastic wire, rod, or flat ribbon or strip of a material. In the embodiments illustrated in FIG. 1A to 3 member 15 is a narrow ribbon structure. Member 15 may be biocompatible with body tissues, for example a shape memory alloy material such as nitinol or the like. In another embodiment member 15 may be tubular or be of a non-solid shape with arterial compressor 18 and support section 19 at opposite ends of member 15.

Device 10R illustrated in FIG. 1B also includes member 15, arterial compressor 18, and support section 19. Arterial compressor 18 and support section 19 may be located at opposite faces of member 15 so as to face in the opposite direction to the arterial compressor 18 and support section 19 on device 10L as seen in FIG. 1B. In the illustrated embodiment, device 10R is a mirror image of device 10L.

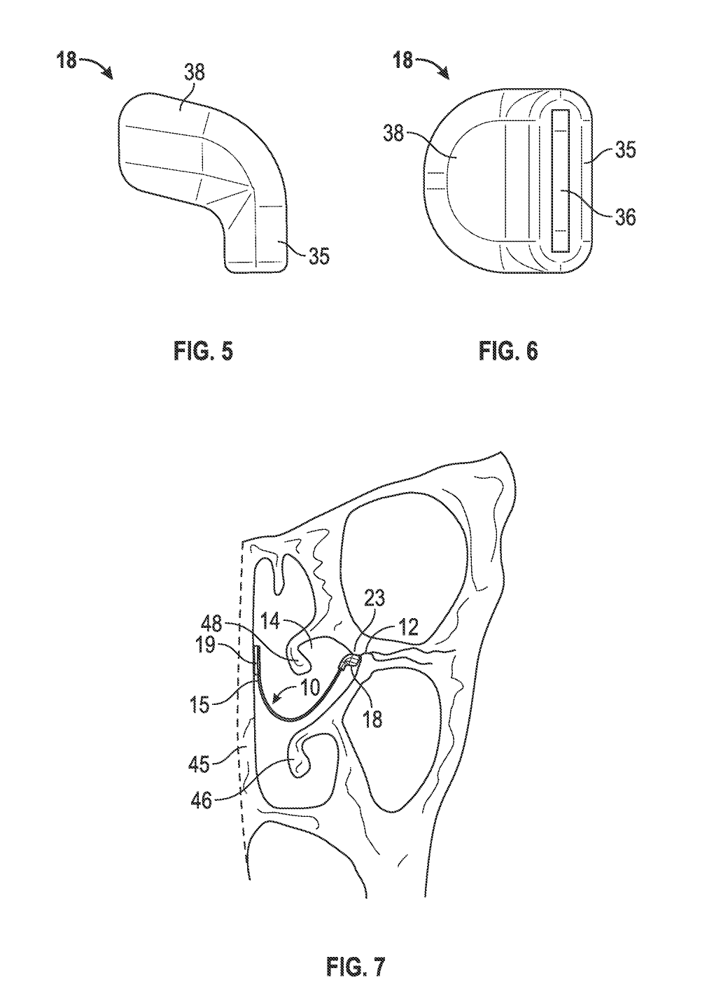

FIGS. 4 to 6 illustrate the separated components of the device 10, and FIG. 7 illustrates device 10 in use. The description in FIGS. 4 to 7 of device 10 apply to both the device 10L and device 10R. FIG. 4 illustrates one embodiment of member 15 in more detail. As illustrated, member 15 includes a central portion 20, arterial end portion 22, and support end portion 24. Arterial end portion 22 and support end portion 24 extend from central portion 20 and may be angled relative to central portion 20. The angle may be in a plane parallel to rectangular shape of central portion 20 and may be relative to a longer side of the rectangle. Central portion 20 may be a relatively thin elongate rectangle or flat strip. Arterial end portion 22 and support end portion 24 may be angled between zero and forty-five degrees relative to central portion 20. In one embodiment, arterial end portion 22 and support end portion 24 are angled twenty degrees relative to central portion 20. In another embodiment arterial end portion 22 and support end portion 24 are angled greater than twenty degrees relative to central portion 20. Angling the arterial end portion 22 and the support end portion 24 may increase visualization when inserting device 10 and when operating on or inspecting the nasal cavity while device 10 is inserted. Angling arterial end portion 22 and support end portion 24 may also allow device 10 to be used with a larger range of different nasal anatomies.

Arterial end portion 22 includes end 26 and may be configured with one or more mounting holes 25 extending through arterial end portion 22. Arterial end portion 22 includes two mounting holes 25 adjacent end 26 in the embodiments shown. Support end portion 24 may include plate 28 and adhesion holes 30. Plate 28 may be an enlarged rectangular or square area.

Member 15 may include guides or locators to help install member 15 into a delivery device or introducer. The guides or locators may protrude from or recede into member 15. Examples include tabs, protrusions, ribs, and slots. In the embodiment depicted, member 15 includes a pair of first guides 32 and a pair of second guides 34. The first guides 32 are tabs and project from one side edge of central portion 20 with one of the pair of guides 32 adjacent arterial end portion 22 and the other of the pair of guides 32 adjacent support end portion 24, each having an ovate shape; the second guides 34 are tabs and project from the opposite side of member 15 with one of the pair of guides 34 projecting from arterial end portion 22 adjacent central portion 20 and the other of the pair of guides 34 projecting from support end portion 24 adjacent central portion 20, each having a triangular shape with a rounded edge.

Guides 32 and 34 may help to place and align device 10 in an introducer or insertion tool configured to insert device 10 into a nasal cavity. For example, guides 32 and 34 may engage slots or groves in an introducer or insertion tool. Such introducers and insertion tools are described in co-pending patent application Ser. No. 13/076,811 filed on Mar. 31, 2011, and co-pending patent application Ser. No. ______ filed concurrently with this application on Aug. 31, 2012 and titled INSERTION TOOL AND INSERTION METHOD FOR ARTERIAL TAMPONADE DEVICE, the contents of which are incorporated herein by reference.

In one embodiment, the length of angled arterial end portion 22 is slightly greater than the length of angled support end portion 24. The overall length of member 15 in one embodiment is around 1.5 inches, while the distance between the second pair of guides 34 is around 0.7 inches, the length of arterial end portion 22 from the inner face of adjacent guide 34 is around 0.42 inches, and the corresponding length of support end portion 24 from the inner face of guide 34 is around 0.38 inches. The width of the central portion 20 and end portion 22 is around 0.14 inches. The thickness of member 15 is around 0.016 inches. As noted above, member 15 may be of a highly resilient or shape memory material such as Nitinol or similar alloy material.

Support section 19 may be a relatively flat section located on support end section 24. Support section 19 may be an integral part of support end section 24 or may be a separate structure. In the embodiment shown, support section 19 is a square pad. In one embodiment the dimensions are of the order of 0.24 by 0.24 inches. Plate 28 may be configured for mounting support section 19 to member 15. Multiple adhesion holes 30 may extend through support end portion 24 to provide for better adhesion of support section 19. In the embodiment depicted in FIG. 4, plate 28 is configured to include adhesion holes 30. The dimensions of support section 18 may substantially match those of plate 28. Support section 19 and plate 28 of member 15 may be of different peripheral shapes in alternative embodiments, such as circular, oval, rectangular, polygonal, or irregular shapes. Alternatively, plate 28 may function directly as the support section.

Arterial compressor 18 may include an attachment end 35 and an engagement end 38. Attachment end 35 may include slot 36. Arterial compressor 18 may be secured over the end 26 of member 15 with end 26 protruding into slot 36, as illustrated in FIG. 2. Engagement end 38 may be a blunted, parabolic, slightly angled overmold designed for applying pressure to tissue above or covering an artery, such as the SPA of the nose. The distal end 40 of engagement end 38 may engage the tissue. Engagement end 38 may extend at a non-parallel angle relative to attachment end 35 and member 15 including arterial end portion 22. In one embodiment, engagement end 38 extends at an angle of around 105 degrees relative to and from attachment end 35 and member 15. In other embodiments, engagement end 38 may be at any angle between 90 degrees to 125 degrees relative to attachment end 35 and member 15. This arrangement angles engagement end 38 slightly outwards or forward from member 15 in the compressed, generally U-shaped configuration illustrated in FIG. 3.

In one embodiment, arterial compressor 18 is injection molded with attachment end 35 engaging over arterial end portion 22 at end 26, so that the molten material used in forming arterial compressor 18 extends through mounting holes 25 during molding to secure arterial compressor 18 to member 15. Alternatively, arterial compressor 18 may be formed separately and secured in place via adhesive placed in slot 36 which flows through mounting holes 25 when end 26 is inserted into slot 36 and subsequently hardens to secure arterial compressor 22 to the member 15. The molding cavity for arterial compressor 18 for right tamponade device 10R is reversed from the molding cavity for device 10L so that engagement end 38 extends in the opposite direction from arterial portion 22 of device 10R than the direction engagement end 38 extends for device 10L. In another embodiment arterial compressor 18 is formed as an integral part of member 15. For example, arterial compressor 18 may be formed integrally with member 15 by stamping. In yet another embodiment arterial end portion 18 includes a first surface and engagement end extends from the first surface.

In one embodiment, the length of engagement end 38 is in the range from around 0.15 to 0.30 inches, the width is in the range from about 0.10 to 0.35 inches, and the thickness is in the range from 0.85 to around 0.10 inches. In another embodiment, engagement end 38 has a length of about 0.16 inches, a width of around 0.175 inches, and a thickness of around 0.09 inches. Arterial compressor 18 may be of different shapes and dimensions in alternative embodiments.

The assembled device 10 may be folded from the expanded, relaxed condition as illustrated in FIG. 1A into a deformed condition by pushing arterial compressor 18 and support section 19 towards one another, or by pushing arterial end portion 22 and support end portion 24 towards one another, forming a generally V-like or U-like looped shape as illustrated in FIG. 3. In the V-like or U-like shapes arterial compressor 18 extends outwardly at an angle and support section 19 faces in the opposite direction relative to arterial compressor 18. As illustrated in FIG. 7, arterial compressor 18 faces in the direction for engaging the tissue over the SPA 12 and support section 19 faces the septum 45 for engaging the septal wall. Both device 10L and device 10R engage the tissue over the SPA 12 and the septal wall in the left and right nasal cavities in the same way.

Arterial compressor 18 and support section 19 may be of any suitable biocompatible material, for example a biocompatible injection molded plastic such as polyamide, polycarbonate, polyolefin, polypropylene, polyurethane or other material. In one embodiment, support section 19 is of a different material from arterial compressor 18, such as polyester felt. Arterial compressor 18 and support section 19 may be rigid or substantially rigid, and either solid or hollow. In alternative embodiments, the material of arterial compressor 18 and support section 19 may be slightly deformable to better match the shape of an opposing surface against which they are pressed on installation. The tissue engaging surfaces of arterial compressor 18 and support section 19 may be designed as non-slip or slip-resistant surfaces, and may be roughened with bumps, indentations, or small openings or pores, for example.

Device 10 may be positioned in a body cavity by means of a standard surgical grasping instrument, via an introducer such as a cylindrical sheath or endoscope, for example as described in co-pending application Ser. Nos. 13/076,811 or ______ cited above, or via other customized delivery systems. Device 10 is designed to be collapsed from its original, relaxed or fully expanded shape as illustrated in FIG. 1 into a compact, folded configuration as illustrated in FIG. 3 for installation purposes, so that it can be moved to a selected region in a body cavity without significantly contacting structures in the path to the desired region. The folded position may also be the U-like or V-like shapes described above. When constructed of a shape memory material such as nitinol, at least a portion of device 10 may be cooled to or below the transformation temperature of the selected shape memory material such that the softer martensitic phase is present prior to folding device 10. Device 10 may then be folded or deformed into the installation U-like or V-like shape while the softer martensitic phase is present in device 10.

The arterial end portion 22 and the support end portion 24 deploy towards the extended, unstressed position of FIG. 1 when member 15 is released from the grasping instrument or introducer. Arterial compressor 18 and support section 19 engage opposite wall regions of the body cavity while the device is still partially folded, so that the partially folded member 15 applies a biasing force pressing arterial compressor 18 and support section 19 against the opposite wall regions. In the embodiment illustrated in FIGS. 1 to 5, device 10 is designed for intranasal placement in a nasal cavity as illustrated in FIG. 7, with the biasing force in the folded member 15 urging the arterial compressor 18 and support section 19 against predetermined opposing areas of the nasal cavity.

In one embodiment, device 10 is positioned in either the right or left nasal cavity so that engagement end 38 of arterial compressor 18 is urged into a depression or area 23 of the SPA foramen including the SPA 12 as illustrated in FIG. 7. Arterial compressor 18 may engage in or near the SPA foramen against the SPA 12 and any of its branches. Support section 19 bears against an area of the nasal septum 45 that opposes area 23. Arterial end portion 22 and support end portion 24 may extend over the inferior turbinate 46 while device 10 is positioned within either the left or right nasal cavity.

Arterial compressor 18 which is designed to engage the tissue surface including one or more blood vessels such as the SPA (or one or more blood vessels in a different body cavity in alternative embodiments) is suitably configured to apply sufficient pressure over an area of the artery to at least substantially occlude blood flow in the artery, and also to minimize trauma to the tissue. Arterial compressor 18 may be positioned to occlude blood flow in one or more arteries or blood vessels, particularly the SPA and branches of that artery.

The internal nasal space may have unique challenges to place and hold a self-retaining device such as device 10. The bony and cartilaginous structures may have cavernous and protruding characteristics, may vary from person to person, and may be affected by a variety of disease states. The SPA foramen is a small, partially hidden osseous foramen. The minute osseous detail of the edges of the SPA foramen and its overall location relative to other structures may be variable. The nasal surfaces are covered by nasal lining and by a respiratory mucus-membrane which is covered with polysaccharides. The nasal lining and polysaccharides may destabilize a self-retaining device. The SPA foramen is under the nasal lining and may not be visible. In the illustrated embodiment, the angle of engagement end 38 and the blunted, parabolic shape of engagement end 38 may help device 10 reach into the SPA foramen and seek a settled and stable position, as illustrated in FIG. 7.

Engagement end 38 may be angled slightly posteriorly and sized to deploy into a predetermined area of a body cavity such as the SPA foramen with its narrowest point and then may taper out to its wider segment so that device 10 may seek and settle into a stable position within the predetermined area such as the SPA foramen. Engagement end 38 may be rounded or bulbous to apply sufficient pressure to occlude an artery such as the SPA. Support section 19 is designed to press against an opposing portion of the body cavity such as the septal wall 45, as illustrated in FIG. 7, when device 10 is properly positioned in the body cavity such as a nasal cavity and released from an insertion tool. Support section 19 may be designed to resist slippage. The surface of support section 19 may be roughened with bumps, indentations, or small openings or pores. Support section 19 may also be designed to aid in the delivery of necessary force, assist in holding device 10 in position, and potentially limit blood flow to a predetermined portion of the body such as the posterior septum.

The distance between the SPA and the septum in typical individuals is approximately 1.2-1.8 cm, with an average of 1.5 cm. The variability is on the septum size and to a lesser extent the skull size of the patient. The dimensions of device 10 in the fully expanded, relaxed condition of FIG. 1 are such that, when positioned and released at the desired location in nasal cavity 14 as in FIG. 7, the device cannot fully expand with member 15 being partially compressed or deformed inwardly from its original straight or partially curved shape so as to apply a biasing force against the opposing cavity surfaces via arterial compressor 18 and support section 19. Device 10 may be of varying lengths. The fully expanded spacing between the ends of arterial compressor 18 and support section 19 is of the order of 30 to 40 mm (1.18 to 1.57 inches). In one embodiment the fully expanded spacing is around 30 mm (1.18 inches). In one embodiment for use in a SPA tamponade, device 10 is designed so that arterial compressor 18 applies pressure of around 2000 gm per sq. cm (around 28 psi) or more to the opposing tissue surface of the lateral nasal wall. In some cases, the device may be designed to apply a significantly higher amount of pressure. Device 10 may be of different sizes and may be provided for different size nasal cavities or for use in different body cavities.

Device 10 is designed to be of relatively low profile when installed in the selected position in the nose. The angled offset between the central portion 20 of member 15 and both arterial end portion 22 and support end portion 24 provides for better visualization of arterial compressor 18 and support section 19 as device 10 is inserted into a nasal cavity, and thus easier and more accurate placement of the device.

When device 10 is placed as illustrated in FIG. 7, arterial compressor 18 and support section 19 engage and apply pressure against the nasal mucosa and bone. Device 10 is of relatively low profile when installed and is placed low in the nose out of the normal surgical field. Engagement end 38 is configured to exert pressure on one or more blood vessels in the area, particularly the underlying SPA 12, so as to temporarily occlude the artery and cut off blood flow to the nasal cavity. This artery is a major blood supply to the nose and occluding it temporarily, for example during endoscopic sinonasal surgeries, reduces intra-operative bleeding and, more importantly, may improve visualization during such procedures. At the conclusion of surgery, device 10 can be removed and discarded, or may be left in place for a time period after surgery if needed.

In the above embodiment, the member 15 of the device 10 is of bendable, resilient metal or plastic material which may be biocompatible. Member 15 is arranged so that when compressed and released, member 15 seeks to return to the original, relaxed and non-compressed state with sufficient tensile strength to ensure stability of device 10 within a nasal cavity and counteract arterial hydrostatic pressure. Substantial opposing force may be necessary to hold a self-retaining device, such as device 10, in position, straddling the septum and natural bony nasal wall. Device 10 uses the bony boundaries of the nasal wall to provide the counter force necessary for stability and pressure. Without sufficient force arterial compressor 18 and support section 19 may slip or destabilize from a surface covered with polysaccharides. Member 15 may be configured in a ribbon-shape to provide such a force. Arterial compressor 18 which engages the area of the nasal wall including the SPA is pressed against the mucosa and underlying arterial wall with sufficient force to occlude or at least substantially occlude the artery.

As noted above, device 10 may be removed and discarded after use. It may be installed prior to surgery in order to reduce blood in the operative field which can interfere with visualization, and may be left in place for a time period after surgery to reduce post-operative bleeding. Device 10 is of low profile so that it is out of the normal surgical field and does not interfere with the surgeon's view of the surgical site. A safety string or tether (not illustrated) may be attached to device 10 and extend out of the nose for assistance in locating, repositioning, and removing device 10 after surgery or after sufficient healing has occurred.

In the above embodiments, one or both arterial compressor 18 and support section 19 may also contain a suitable medication and may slowly release the medication during and after surgery. For example, the tissue engaging surfaces of arterial compressor 18 and support section 19 may have a drug-eluting coating of a material which elutes a drug onto the adjacent tissue, such as a sinus treatment drug, an infection or inflammation combating drug, or a drug which reduces bleeding. The medication is designed to be gradually released from arterial compressor 18 or support section 19 onto the adjacent cavity wall surfaces over time.

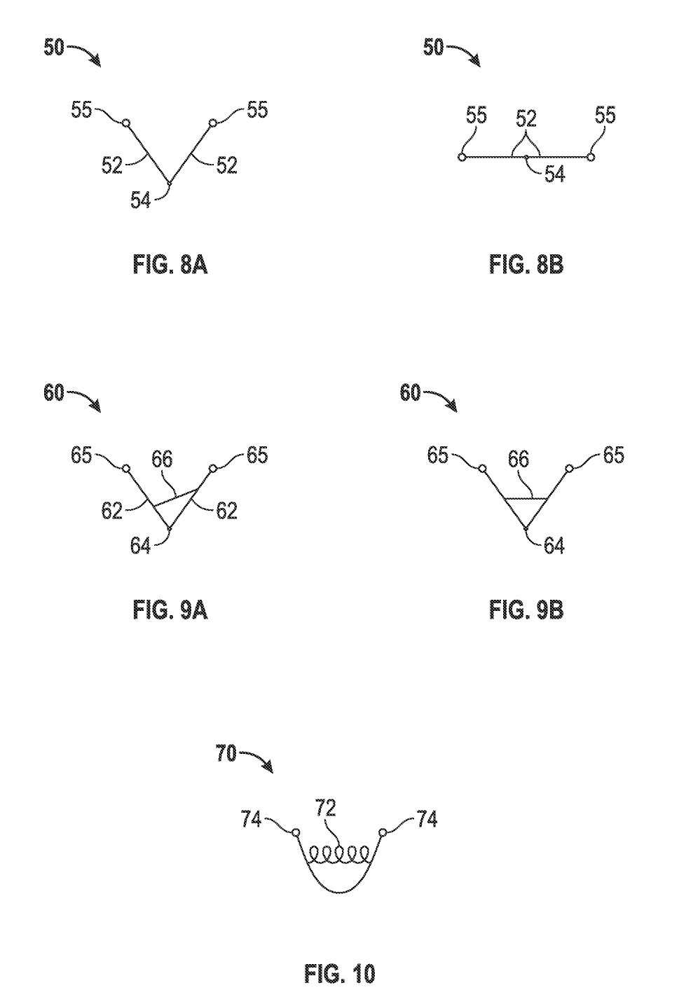

FIGS. 8A and 8B illustrate an intranasal arterial tamponade device 50 according to another embodiment. Device 50 comprises a pair of legs 52 which may be of flexible or inflexible material and which are joined together at a “knee” type of joint or hinge 54. The device is movable between an initial, generally V-shaped configuration as in FIG. 8A and a straightened, operative position as in FIG. 8B in which the pressure applying pads or feet 55 at the ends of the legs are moved outward and maintain a locked and stressed position, creating an outward force on the feet of the device which are pressed against opposing portions of the nasal cavity, as in the previous embodiment.

FIGS. 9A and 9B illustrate a variation of the embodiment of FIGS. 8A and 8B in which tamponade device 60 also has a pair of legs 62 joined at a hinge 64 and having pressure applying feet 65 at the ends of the legs. In this embodiment, a hinged post 66 extending between the legs is movable from a first, inoperative position as in FIG. 9A into an operative, locked position as in FIG. 9B in which the legs are urged apart to apply outward force to feet 65.

FIG. 10 illustrates another alternative intranasal arterial tamponade device 70 which is generally U-shaped or V-shaped and has legs which are urged apart by a biasing member 72 extending between opposing portions of the legs and urging them into a more open position so that biasing force is applied by feet 74 against opposing portions of the nasal cavity. The biasing member may be a spring, as illustrated in FIG. 10, or may be an elastomeric member, an accordion-like expandable member, a bellows, a balloon, or the like.

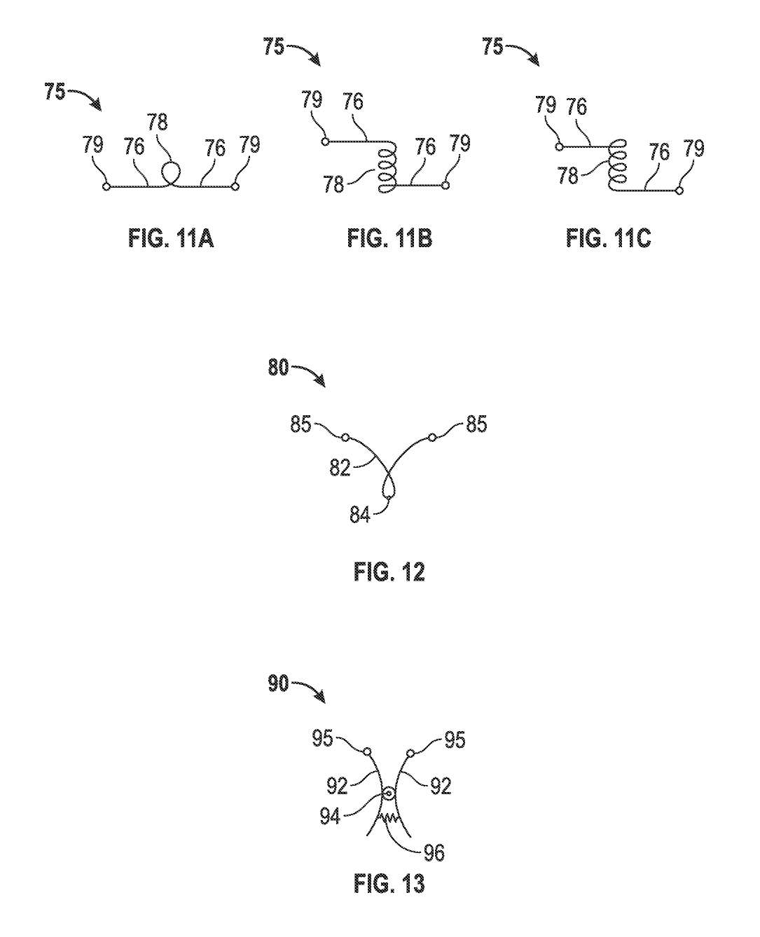

FIG. 11A to 11C illustrate another alternative embodiment of an intranasal arterial tamponade device 75 in which legs 76 are connected by a “mousetrap”-like spring 78. FIG. 11C illustrates the device in a compressed condition in which the feet 79 are urged inwardly into spring 78, while FIGS. 11A and 11B are side and top views, respectively, illustrating the device in an expanded condition in which feet 79 apply pressure to opposing portions of the nasal cavity.

FIG. 12 illustrates another alternative intranasal tamponade device 80 which has a pair of legs 82 connected by a reverse-springing hinge 84 at the apex of the V-shape, creating outer pressure at the feet 85 when compressed.

FIG. 13 illustrates another alternative intranasal tamponade device 90 which has a pair of arcuate legs 92 hinged together via hinge 94 at a central portion along the length of each leg. First ends of the legs on one side of the hinge have feet 95 for applying pressure to opposing portions of the nasal cavity. A ratchet device 96 extends between opposite portions of the legs on the opposite side of hinge 94. Contraction of ratchet device 96 pivots the legs about hinge 94 so as to urge the feet 95 apart.

The devices of FIGS. 8A to 13 may be made of various types of metal and plastic materials suitable for use in the body. One of feet 55, 65, 74, 79, 85, and 95 may be configured similarly to arterial compressor 18, while the other of feet 55, 65, 74, 79, 85, and 95 may be configured similarly to support section 19.

In addition to use during surgery for improved visualization purposes, the above devices may also be used for other purposes. For example, the tamponade device may be left in place after surgery for reduction of post-operative bleeding and also as a post-operative stenting device. The tamponade device may also be used as an alternative to lengthy periods of nasal packing, invasive arterial embolization, or permanent vascular ligation procedures for treating chronic nose bleeds or refractory epitaxis.

Although the above embodiments describe use of an arterial tamponade device in the nasal cavity, a similar device of appropriately modified dimensions may be used in other regions of the body to temporarily reduce or eliminate blood flow through an artery if needed during surgery, following surgery or other trauma, or due to a medical condition which causes excessive bleeding. The dimensions of the device may be adjusted as appropriate so that arterial compressor 18 and support section 19 bear against opposing regions of a selected body cavity with one pad pressing against a part of the cavity wall including one or more arteries or other blood vessels so as to occlude or at least substantially occlude the vessel or vessels and reduce or cut off blood flow.

The above description of the disclosed embodiments is provided to enable any person skilled in the art to make or use the invention. Various modifications to these embodiments will be readily apparent to those skilled in the art, and the generic principles defined herein can be applied to other embodiments without departing from the spirit or scope of the invention. Thus, the invention is not intended to be limited to the embodiments shown herein but is to be accorded the widest scope consistent with the principles and novel features disclosed herein.

We claim:

1. A tamponade device for engagement in a human nasal cavity and applying pressure against a predetermined area of a lateral nasal wall including the sphenopalatine artery (SPA), comprising:
an elongate member having
a central portion,
an arterial end portion extending from one end of the central portion, the arterial end portion including a first surface, and
a support end portion extending from an opposite end of the central portion;
an arterial compressor located at the arterial end portion, the arterial compressor having
an engagement end extending from the first surface, the engagement end being configured to engage a depression in the SPA foramen which houses the SPA; and
a support section located at the support end portion, the support section having a relatively flat tissue engaging surface configured to engage an opposing portion of the septum.

2. The tamponade device of claim 1, wherein the arterial end portion extends at an angle between zero and forty-five degrees relative to the central portion and the support end portion extends at an angle between zero and forty-five degrees relative to the central portion.

3. The tamponade device of claim 1, wherein the member includes a pair of first guides located on an edge of the central portion with one of the pair of first guides located adjacent the arterial end portion and the other of the pair of first guides located adjacent the support end portion and a pair of second guides with one of the pair of second guides located on an edge of the arterial end portion adjacent the central edge portion and the other of the pair of second guides located on an edge of the support end portion adjacent the central edge portion.

4. The tamponade device of claim 1, wherein the elongate member comprises a length of biocompatible, resilient material.

5. The tamponade device of claim 4, wherein the biocompatible material is nitinol.

6. The tamponade device of claim 4, wherein the arterial compressor and support section each comprise a biocompatible material.

7. The tamponade device of claim 1, wherein the arterial compressor is of injection molded plastic material.

8. The tamponade device of claim 7, wherein the plastic material is nylon.

9. The tamponade device of claim 7, wherein the arterial end portion includes a mounting hole and the arterial compressor is injection molded with an attachment end engaging over the arterial end portion so that the molten plastic material extends through the mounting hole during molding to secure the arterial compressor to the member.

10. The tamponade device of claim 1, wherein the arterial compressor includes an attachment end and the engagement end extends from the attachment end at an angle between ninety to one-hundred and twenty five degrees relative to the attachment end.

11. The tamponade device of claim 10, wherein the engagement end extends at an angle of one-hundred and five degrees relative to the attachment end.

12. The tamponade device of claim 1, wherein the engagement end is a blunted, parabolic, slightly angled overmold configured for applying pressure to tissue above the SPA.

13. A tamponade device for engagement in a human nasal cavity and applying pressure against a predetermined area of a lateral nasal wall including the sphenopalatine artery (SPA), comprising:
an elongate member having
a central portion,
an arterial end portion extending from one end of the central portion at an angle between zero and forty-five degrees relative to the central portion, and
a support end portion extending from an opposite end of the central portion at an angle between zero and forty-five degrees relative to the central portion;
an arterial compressor located at the arterial end portion;
a support section located at the support end portion; and
the elongate member being configured for bending into a generally U-shaped or V-shaped, compressed condition with the compressed elongate member being of predetermined dimensions for placement in a nasal cavity on one side of the nasal septum and being configured to bias the arterial compressor and support section outwards towards an expanded condition when released at a selected location in the nasal cavity until the arterial compressor and support section engage and apply pressure to the opposing portions of the SPA foramen and septum, respectively, in a partially expanded condition of the elongate member.

14. The tamponade device of claim 13, wherein the elongate member includes a pair of first guides located on an edge of the central portion with one of the pair of first guides located adjacent the arterial end portion and the other of the pair of first guides located adjacent the support end portion.

15. The tamponade device of claim 14, wherein the elongate member includes a pair of second guides with one of the pair of second guides located on an edge of the arterial end portion adjacent the central edge portion and the other of the pair of second guides located on an edge of the support end portion adjacent the central edge portion.

16. The tamponade device of claim 14, wherein the elongate member includes a pair of second guides with one of the pair of second guides located on an edge of the arterial end portion adjacent the central edge portion and the other of the pair of second guides located on an edge of the support end portion adjacent the central edge portion with the first guides and the second guides being located on opposing edges of the member.

17. The tamponade device of claim 16, wherein the pair of first guides and the pair of second guides are tabs extending from their respective edge locations.

18. The tamponade device of claim 13, wherein the elongate member comprises a length of biocompatible, resilient material.

19. The tamponade device of claim 18, wherein the biocompatible material is nitinol.

20. The tamponade device of claim 18, wherein the arterial compressor and support section each comprise a biocompatible material.

21. The tamponade device of claim 13, wherein the support end portion includes a plate with a rectangular or square shape and the support section includes a matching rectangular or square shape with a uniform thickness having an outer, flat face comprising a tissue engaging face.

22. The tamponade device of claim 21, wherein the plate is configured with a plurality of adhesion holes for securing the support section to the support end portion.

23. The tamponade device of claim 21, wherein the tissue engaging face includes a roughened, slip resistant surface texture.

24. The tamponade device of claim 13, wherein the engagement end is a blunted, parabolic, slightly angled overmold configured for applying pressure to tissue above the SPA.

25. The tamponade device of claim 13, wherein the arterial end portion extends from the central portion at a twenty degree angle relative to the central portion and the support end portion extends from the central portion at a twenty degree angle relative to the central portion.

26. The tamponade device of claim 13, wherein the member is 1.5 inches long, the length of the arterial end portion is 0.42 inches, and the length of the support end portion is 0.38 inches.

27. A tamponade device for engagement in a human nasal cavity and applying pressure against a predetermined area of a lateral nasal wall including the sphenopalatine artery (SPA), comprising:
an elongate member with a narrow ribbon structure, the member having
an arterial end portion,
a support end portion,
a pair of first guides protruding from an edge of the member, and
a pair of second guides protruding from an opposite edge of the member;
an arterial compressor located at the arterial end portion;
a support section located at the support end portion; and
the first guides and the second guides being configured to help place and align the tamponade device into an installation tool or introducer.

28. The tamponade device of claim 27, wherein the first guides have an ovate shape and the second guides have a triangular shape with a rounded edge.

29. The tamponade device of claim 27, wherein the member comprises a length of biocompatible, resilient material.

30. The tamponade device of claim 29, wherein the biocompatible material is nitinol.

31. A tamponade device for engagement in a human nasal cavity and applying pressure against a predetermined area of a lateral nasal wall including the sphenopalatine artery (SPA), comprising:
an elongate member with a narrow ribbon structure, the member having
a central portion,
an arterial end portion extending from the central portion at an angle between zero and forty-five degrees relative to the central portion,
a support end portion extending from the central portion at an angle between zero and forty-five degrees relative to the central portion and opposing the arterial end portion,
a pair of first guides located on an edge of the central portion with one of the pair of first guides located adjacent the arterial end portion and the other of the pair of first guides located adjacent the support end portion, and
a pair of second guides with one of the pair of second guides located on an edge of the arterial end portion adjacent the central edge portion and the other of the pair of second guides located on an edge of support end portion adjacent the central edge portion with the first guides and the second guides being located on opposing edges of the elongate member;
an arterial compressor located at the arterial end portion, the arterial compressor having
an engagement end extending at an angle between ninety to one-hundred and twenty five degrees relative the elongate member in a direction generally away from the central portion, the engagement end being blunted with a parabolic shape; and
a support section located at the support end portion, the support section having a relatively flat tissue engaging surface.

32. The tamponade device of claim 31, wherein the elongate member comprises a length of biocompatible, resilient material.

33. The tamponade device of claim 32, wherein the biocompatible material is nitinol.

34. The tamponade device of claim 31, wherein the arterial compressor and support section each comprise a biocompatible material.

35. The tamponade device of claim 31, wherein the support section includes a plate with a rectangular or square shape with a uniform thickness.

36. The tamponade device of claim 31, wherein the arterial end portion includes an end with mounting holes and the arterial compressor is injection molded with an attachment end engaging over the arterial end portion so that a molten plastic material of the arterial compressor extends through the mounting hole during molding to secure the arterial compressor to the elongate member.

37. The tamponade device of claim 31, wherein the engagement end extends at an angle of one-hundred and five degrees relative to the elongate member.

38. The tamponade device of claim 31, wherein the arterial end portion extends from the central portion at a twenty degree angle relative to the central portion and the support end portion extends from the central portion at a twenty degree angle relative to the central portion.

39. The tamponade device of claim 31, wherein the pair of first guides are tabs with an ovate shape and the pair of second guides are tabs with a triangular shape with a rounded edge.
